(19) United States
(12) Patent Application Publication
(10) Pub. No.: US 2013/0338200 A1
(43) Pub. Date: Dec. 19, 2013

(54) PHARMACEUTICAL DOSAGE FORMS OF TIZANIDINE AND ADMINISTRATION ROUTES THEREOF

(71) Applicant: Massimiliano BORSA, Vimodrone (IT)

(72) Inventor: Massimiliano BORSA, Vimodrone (IT)

(73) Assignee: MDM SpA, Milano (IT)

(21) Appl. No.: 13/495,555

(22) Filed: Jun. 13, 2012

(51) Int. Cl. A61K 31/433; A61P 25/14

(52) U.S. Cl. 514/362

(57) ABSTRACT

A pharmaceutical composition in liquid dosage form which contains a tizanidine hydrocloride aqueous solution suitable for parenteral, intranasal and oral administration.

### BACKGROUND OF THE INVENTION

1. Field of the Invention

The present invention relates to a pharmaceutical composition in liquid dosage form which contains a tizanidine hydrocloride aquous solution suitable for parenteral, intranasal and oral administration.

2. Description of the Background

Tizanidine, 5-chloro-N-(4,5-dihydro-1H-imidazol-2-yl)-2,1,3-benzothia-diazol-4-amine, is a α₂-adrenergic agonist structurally related to clonidine. It is a centrally acting skeletal muscle relaxant that acts mainly at spinal and supraspinal levels to inhibit excitatory interneurones. It is indicated for the symptomatic relief of spasticity associated with multiple sclerosis or spinal cord injury or disease or painful muscle spasm.

The compound tizanidine, as hydrochloride, is included in Japan and US Pharmacopoeias and it is approved and marketed worldwide, as base, in the form of 2 or 4 mg tablets. As such, tizanidine is usually well and rapidly absorbed by oral route, but it undergoes extensive first-pass effect which inactivates metabolites.

The pharmacokinetic profile of tizanidine, Tₘₐₓ 1-2 h, T_{1/2z} 1-2 h, suggested to perform slow-release dosage forms as in US 2008 0214629A1 or WO 2008 047208A1, but, on the other hand, the entity of the first-pass effect, suggested to replace oral absorption with different routes of administration.

The following are known pharmaceutical formulations containing tizanidine: trans-dermal formulations are described in DK 175982B1, buccal spray formulations are described in WO 2004 019905A1, buccal and sublingual formulations are described in WO 2004 043431A1 and in US 2004 0122065A1. The aim of these formulations is to increase the onset of action of tizanidine as well as its bioavailability and to reduce the relative hepatic first-pass effect.

The following pharmacological reports describe the administration of tizanidine by intramuscular (IM) or intrathecal route to assess its mechanism of action and compare its potency with standard drugs: J. Neurosurg 2004, 101(4):641-7; J. Pharmacol Sci. 2005, 99(1):52-60; Anesthesiology 2003, 98(6):1480-3; Anesth. Analg. 2003, 96(3):776-82; Anesth. Analg. 2001, 93(5):1310-5.

### SUMMARY OF THE INVENTION

It is the object of the present invention a pharmaceutical composition in liquid dosage form which contains as active principle the α₂-adrenergic agonist compound Tizanidine.

According to the invention it has been found that tizanidine, in the form of the corresponding hydrochloride, is soluble in water at concentrations and pH suitable for systemic administration, specifically for parenteral, intranasal and oral administration, and that these aqueous solutions of tizanidine hydrochloride may be normally stored and, in addition, easily sterilized by heat so that the use of preservative agents can be, when preferred, avoided.

Accordingly, in one embodiment, the present invention is directed to a pharmaceutical composition suitable for oral, parenteral or intranasal administration comprising tizanidine hydrocloride dissolved in a medium selected from the group consisting of water, an aqueous saline solution and an aqueous buffer solution, wherein the composition has a pH of 4.5 to 7.5.

In another embodiment, the present invention is directed to a pharmaceutical composition as described above, which is sterilized.

In another embodiment, the present invention is directed to a pharmaceutical composition as described above, which is sterilized by heat treatment.

In another embodiment, the present invention is directed to a pharmaceutical composition as described above, further comprising at least one preservative agent.

In another embodiment, the present invention is directed to a pharmaceutical composition as described above, further comprising at least one flavoring agent, tonicity agent, hyperbaric agent or absorption enhancer agent.

In another embodiment, the present invention is directed to pharmaceutical composition as described above, which has a pH of from 4.5 to 6.5.

In another embodiment, the present invention is directed to a pharmaceutical composition as described above, having a concentration of from 0.10 to 2.00 mg/mL of tizanidine base.

In another embodiment, the present invention is directed to a pharmaceutical composition as described above, having a concentration of from 0.10 to 2.00 mg/mL of tizanidine base and a pH of from 4.5 to 6.5.

In another embodiment, the present invention is directed to a pharmaceutical composition as described above, further comprising at least one tonicity agent or a hyperbaric agent.

In another embodiment, the present invention is directed to a pharmaceutical composition as described above, having a concentration of from 0.10 to 1.00 mg/mL of tizanidine base.

In another embodiment, the present invention is directed to a pharmaceutical composition as described above, having a concentration of from 0.10 to 1.00 mg/mL of tizanidine base and a pH of from 4.5 to 6.5.

In another embodiment, the present invention is directed to a pharmaceutical composition as described above, further comprising at least one tonicity agent or a hyperbaric agent.

In another embodiment, the present invention is directed to a pharmaceutical composition as described above, further comprising at least one preservative agent selected from the group consisting of benzyl chloride, methylparahydroxybenzoate, ethylparahydroxybenzoate and propylparahydroxybenzoate and at least one flavoring agent selected from the group consisting of aroma consisting of citrus, eucalyptol, eucalyptus oil and peppermint.

In another embodiment, the present invention is directed to a pharmaceutical composition as described above, further comprising at least one tonicity agent and/or hyperbaric agent.

In another embodiment, the present invention is directed to a pharmaceutical composition as described above, having a concentration of from 0.10 to 45.0 mg/mL of tizanidine base.

In another embodiment, the present invention is directed to a pharmaceutical composition as described above, having a concentration of from 0.10 to 45.00 mg/mL of tizanidine base and a pH of from 4.5 to 6.5, and wherein the composition contains saline, glucose or a mixture thereof.

In another embodiment, the present invention is directed to a pharmaceutical composition as described above, having a concentration of from 10.0 to 45.0 mg/mL of tizanidine base, especially 10 to 40 mg/mL.

In another embodiment, the present invention is directed to a pharmaceutical composition as described above, having a concentration of from 20.0 to 45.0 mg/mL of tizanidine base and a pH of from 4.5 to 7.4, and wherein the composition further comprises at least one absorption enhancer agent selected from the group consisting of chitosan, methylpyrrolidone and cholic acid.

In another embodiment, the present invention is directed to a pharmaceutical composition as described above, which contains chitosan.

In another embodiment, the present invention is directed to a method of making the pharmaceutical composition described above, comprising dissolving tizanidine hydrocloride in the medium.

In another embodiment, the present invention is directed to a method of delivering tizanidine, comprising orally, parenterally or intranasally administering the pharmaceutical composition of Claim 1 to a subject.

In another embodiment, the present invention is directed to a method of treating muscle spasms, comprising administering an effective amount of the pharmaceutical composition as described above to a subject in need thereof.

In another embodiment, the present invention is directed to the method as described above, wherein the pharmaceutical composition is administered orally, parenterally or intranasally.

### DESCRIPTION OF THE FIGURE

FIG. 1 shows the mean time courses up to 12 hours post-dose. R=Sirdalud tablet; T1=1*70 μL/puff by intranasal route; T2=2*70 μL/puff by intranasal route.

### DETAILED DESCRIPTION OF THE INVENTION

Pharmaceutical compositions of tizanidine in liquid form, such as the aqueous solutions of the present invention, offer different advantages: they can be administered by parenteral route, i.e. intramuscular (IM), intravenous (IV), or intrathecal route, thus overcoming the first-pass effect; they can be administered by oral route (PO) to fulfill the individual required dose according to the specific pathology; they can be administered by intranasal route (IN) to increase the bioavailability overcoming the hepatic first-pass effect and/or to shorten the time to peak.

The transmucosal nasal delivery, represents a delivery option for drugs with limited oral bioavailability due to the degradation in the intestinal tract, such as proteins, or hepatic first-pass metabolism, and is also a convenient alternative to intravenous or intramuscular drug administration. The considerable blood flow, actually responsible for breath conditioning, benefits from the efficient systemic drug uptake and provides direct access to the systemic circulation for transmucosal adsorbed drug.

In accordance with the specific administration route to be used, the composition of the invention may also contain suitable, commonly used agents such as pH buffer, preservative, flavoring, absorption enhancer and hyperbaric agents.

Among commonly used suitable pH buffer agents, aqueous buffer acetate and aqueous buffer phosphate solutions are preferred. Among the commonly used preservative agents, benzyl chloride, methyl-, ethyl- and propylparahydroxybenzoate are preferred. Among commonly used water soluble flavoring agents citrus, preferably orange, eucalyptol, eucalyptus oil and peppermint are preferred. Among commonly used absorption enhancer agents chitosan, methylpyrrolidone and cholic acid are preferred. Suitable tonicity of solutions may be obtained by addition of saline or salt solutions.

The amount of tizanidine, as base, contained in the composition of the invention, which can be daily administered to a patient, may vary in a large range and depends on various factors, such as the pathology to be treated, the route of administration and relative bioavailability, the age and conditions of the patient.

In addition, the pH of the composition may range from 4.5 to 7.5. This range, and all other ranges described herein, includes as specific values and subranges therebetween, such as 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3 and 7.4.

The composition of the invention may contain from 0.10 to 45 mg/mL of tizanidine base. This range, and all other ranges described herein, includes all specific values and subranges therebetween, such 0.2, 0.5, 1, 2, 5, 10, 15, 20, 25, 30, 35 and 40 mg/mL. For intramuscular administration, a preferred range is 0.1 to 2.0 mg/mL. For intrathecal administration, a preferred range is 0.1 to 1.0 mg/mL. For oral administration, a preferred range is 0.1 to 45 mg/mL. For use as an intranasal spray, a preferred range is from 10.0 to 40.0 mg/mL, especially 20 to 40 mg/mL.

For oral administration, the daily dose of tizanidine, as base, is in the range from 2.00-16.00 mg/day. This range includes all specific values and subranges therebetween, such as 4, 6, 8, 10, 12 and 14 mg/day. The pH of the oral aqueous pharmaceutical composition may vary from 4.5 to 6.5 and, usually, preservatives, such as benzyl chloride, methyl-, ethyl- and propylparahydroxybenzoate or similar products and flavoring as citrus, preferably orange, or eucalyptol, eucalyptus oil, peppermint aroma and similar products may also be added thereto.

For parenteral administration, the daily dose of tizanidine, as base, is generally included in the range 0.10-8.00 mg/day, more precisely, in the range from 2.00 to 8.00 mg/day for the intramuscular administration and from 0.10 to 1.00 .mg/day for intrathecal administration. These ranges includes all specific values and subranges therebetween, such as 0.2, 0.5, 0.75, 1.0, 1.5, 2.0, 3.0, 4.0, 5.0, 6.0 and 7.0 mg/day. The pH value of the parenteral aqueous pharmaceutical composition, precisely for intramuscular administration, may vary from 4.6 to 5.8 and for intrathecal administration it may vary from 5.0 to 5.5. Parenteral aqueous pharmaceutical compositions for intrathecal administrations may also contain suitable hyperbaric agents, optionally containing glucose, as enhancer of the drug penetration.

For intranasal administration, the daily dose of tizanidine, as base, is generally in the range 6.00-12.00 mg/day. This range includes all specific values and subranges therebetween, such as 7.0, 8.0, 9.0, 10.0 and 11.0 mg/day. The pH value of the intranasal aqueous pharmaceutical composition may vary from 4.8 to 7.4. Tizanidine intranasal administrations may contain absorption enhancer agents such as chitosan, methylpyrrolidone or cholic acid and preservative agents such as benzyl chloride, methyl-, ethyl- and propylparahydroxybenzoate or similar products and flavoring compounds as citrus, preferably orange, eucalyptol, eucalyptus oil or peppermint aroma and similar products.

The compositions containing tizanidine hydrochloride aqueous solutions of the present invention, that is the compositions suitable for oral, parenteral and intranasal administration, proved to be well tolerated at the administration site, and showed themselves to be effective for relief both of muscle spasm and of multiple sclerosis and neuronal spasticity. They can be administered over a wide range of doses according to the pathology, route of administration, bioavailability, and peak time.

As discussed above, the pharmaceutical composition described herein may be prepared by dissolving tizanidine hydrocloride in the medium.

The composition of the invention may be used to deliver tizanidine by orally, parenterally or intranasally administering the pharmaceutical composition of to a subject. A preferred subject is a human.

The composition of the invention may also be used to treat muscle spasms, by administering an effective amount of the pharmaceutical composition as described above to a subject in need thereof. A preferred subject is a human. The subject may be suffering from spasticity associated with multiple sclerosis or spinal cord injury or disease or painful muscle spasms.

Examples of specific formulations and physico-chemical properties properly tailored for the different route of administration are described below.

### EXAMPLES

Having generally described this invention, a further understanding can be obtained by reference to certain specific examples which are provided herein for purposes of illustration only and are not intended to be limiting unless otherwise specified.

### Parenteral Solution for Urgency or Intrathecal Administration:

Intramuscular administration: solution containing 2.29 mg/mL of tizanidine HCl, equal to 2.00 mg of tizanidine base, without any preservative.

Intrathecal administration: solution containing 0.1145-1.45 mg/mL of tizanidine HCl, equal to 0.10-1.00 mg of tizanidine base, without any preservative, both normal and hyperbaric solution.

### Peroral Solutions:

Aqueous solution at 2.29 mg/mL suitable to be dispensed as 10/20 drops or 0.5/1 mL solution to be diluted with water, equal to 1.00 or 2.00 mg of tizanidine base.

Aqueous solution at 4.58 mg/mL suitable to be dispensed as 10/20 drops or 0.5/1 mL solution to be diluted with water, equal to 2.00 or 4.00 mg of tizanidine base.

Aqueous solution at 9.16 mg/mL suitable to be dispensed as 10/20 drops or 0.5/1 mL solution to be diluted with water, equal to 4.00 or 8.00 mg of tizanidine base.

Drinkable solutions at 0.5725 mg/mL suitable to be dispensed as single dose bottle or sachet containing 4.00 or 8.00 mL, equal to 2.00 or 4.00 mg of tizanidine base.

Drinkable solutions at 2.29 mg/mL suitable to be dispensed as single dose bottle or sachet containing 4.00 or 8.00 mL, equal to 8.00 or 16.00 mg of tizanidine base.

### Intranasal Solutions:

Aqueous solutions, with or without flavor, at 22.90 mg/mL suitable to be dispensed as two 50 μL puff by 0.05 mL snap-on pump, equal to 2 mg of tizanidine base.

Aqueous solutions, with or without flavor, at 45.80 mg/mL suitable to be dispensed as two 50 μL puff by 0.05 mL snap-on pump, equal to 4.00 mg of tizanidine base.

### Preparation of Composition for Intramuscular Injections:

Tizanidine hydrochloride can be dissolved in water, saline or in buffer solution at pH range 4.5-6.5, filtered, dispensed in ampoules, and sterilized by heating in autoclave according to the common methods, for instance at 121° C. for 15 minutes (see Ph. Eur. 5.1.1).

The terminal sterilisation by heat is preferable than the 0.22 μm filtration because it does not need any preservative agent. Simple water solutions are hypotonic due to the low concentration of tizanidine hydrochloride (1.145 or 2.29 mg/mL), therefore it is better to adjust tonicity with saline or buffers to increase local tolerability.

### Example 1

### 2 mL Vial Containing

| | |
|---|---|
| Tizanidine•HCl | 2.29 mg |
| Saline to | 2.00 mL |

Light yellow and clear solution, pH 5.0.

### Example 2

### 2 mL Vial Containing

| | |
|---|---|
| Tizanidine•HCl | 2.29 mg |
| Acetate buffer pH 4.6 | 2.00 mL |
| (Ph. Eur.4001400 diluted 1 to 6 | |
| with water) to | |

Light yellow and clear solution, pH 4.6.

### Example 3

1 mL vial containing:

| | |
|---|---|
| Tizanidine•HCl | 1.145 mg |
| Sodium phosphate buffer pH 5.8 | 1.00 mL |
| (Ph. Eur. 4002100 diluted 1 to 6 with | |
| water) to | |

Light yellow and clear solution, pH 5.8.

### Preparation of Composition for Intrathecal Injections:

The parenteral administration in the spinal cord may increase the local inhibitory effect on the neurones, and the analgesic effect of the compound. Animal data suggest an equivalent activity of tizanidine and clonidine, by local administration, therefore the concentration for injectables ranges between 0.10 and 1.00 mg/mL. According to the experience of anaesthetic drugs, a hyperbaric solution containing glucose could improve the penetration of the drug into the spinal cord and, by consequence, its efficacy. The formulations for this administration route are single dose, preservative-free solution, sterilized by heat.

### Example 4

### 1 mL Vial Containing

| | |
|---|---|
| Tizanidine•HCl | 0.1145 mg |
| Saline to | 1.00 mL |

Clear or almost clear solution, pH 5.4
This formulation is ready for use.

### Example 5

### 1 mL Vial Containing

| | |
|---|---|
| Tizanidine•HCl | 1.145 mg |
| Saline to | 1.00 mL |

Light yellow, clear solution, pH 5.0
This formulation can be diluted with saline accordingly to the prescribed dose.

### Example 6

### 1 mL Vial Containing

| | |
|---|---|
| Tizanidine•HCl | 0.1145 mg |
| 5% Glucose solution to | 1.00 mL |

Clear or almost clear solution, pH 5.5
This formulation is ready for use and is not to be diluted.

### Example 7

### 1 mL Vial Containing

| | |
|---|---|
| Tizanidine•HCl | 1.145 mg |
| 5% Glucose solution to | 1.00 mL |

Light yellow, clear solution, pH 5.0
This formulation is ready for use and is not to be diluted.

### Preparation of Compositions for Oral Solutions:

Tizanidine.HCl aqueous solutions are, according to the concentration, yellow coloured, transparent, pH 4.8, and without any taste. The solutions require to be preserved from yeast and bacterial grow and the range of natural pH, i.e. between 4.5 and 5.0, suggests, as first choice, methyl p-hydroxybenzoate or a mixture of hydroxybenzoates as preservative since these are effective and chemically stable in this range of pH, and do not react with tizanidine.

Tizanidine solutions are tasteless, and to avoid a second accidental administration, which may happen using multidose forms, a flavor to remind the previous administration is added to the formulations. The single dose unit avoids any risk of overdose.

### Example 8

### Multidose Glass or Aluminium Container Holding

| | |
|---|---|
| Tizanidine•HCl | 2.29 mg |
| Methyl p-hydroxybenzoate | 1.00 mg |
| Eucalyptol | 4.00 mg |
| 95% Ethanol | 0.10 mL |
| Water to | 1.00 mL |

Yellow, clear solution, pH 4.8

The single doses, 0.5 mL, containing 1.00 mg of tizadine base, or 1 mL, containing 2.00 mg of Tizanidine base, are dispensed by a dropper or a graduated syringe or a pump.

### Example 9

### Multidose Glass or Aluminium Container Holding

| | | |
|---|---|---|
| Tizanidine•HCl | 4.58 | mg |
| Methyl p-hydroxybenzoate | 1.00 | mg |
| Citrus flavor | 10.00 | mg |
| 95% Ethanol | 0.10 | mL |
| Water to | 1.00 | mL |

Yellow, clear solution, pH 4.9

The single doses, 0.5 mL, containing 2.00 mg of tizadine base, or 1 mL, containing 4.00 mg of Tizanidine base, are dispensed by a dropper or a graduated syringe or a pump.

### Example 10

### Multidose Glass or Aluminium Container Holding

| | | |
|---|---|---|
| Tizanidine•HCl | 9.16 | mg |
| Methyl p-hydroxybenzoate | 1.00 | mg |
| Peppermint aroma | 1.00 | mg |
| 95% Ethanol | 0.05 | mL |
| Water to | 1.00 | mL |

Yellow, clear solution, pH 4.7

The single dose, 0.5 mL, containing 4.00 mg of tizanidine base, or 1 mL containing 8.00 mg of tizanidine base, are dispensed by a dropper or a graduated syringe or a pump.

### Example 11

### A Single Dose Sealed Aluminium Sachet Containing

| | | |
|---|---|---|
| Tizanidine•HCl | 2.29 | mg |
| Methyl p-hydroxybenzoate | 6.00 | mg |
| Citrus flavor | 40.00 | mg |
| 95% Ethanol | 0.40 | mL |
| Water to | 4.00 | mL |

Light yellow, clear solution, pH 5.6.

### Example 12

### A Single Dose Sealed Aluminium Sachet Containing

| | | |
|---|---|---|
| Tizanidine•HCl | 4.58 | mg |
| Methyl p-hydroxybenzoate | 12.00 | mg |
| Citrus flavor | 80.00 | mg |
| 95% Ethanol | 0.80 | mL |
| Water to | 8.00 | mL |

Light yellow, clear solution, pH 5.6.

### Example 13

### A Single Dose Sealed Aluminium Sachet Containing

| | | |
|---|---|---|
| Tizanidine•HCl | 9.16 | mg |
| Methyl p-hydroxybenzoate | 6.00 | mg |
| Citrus flavor | 40.00 | mg |
| 95% Ethanol | 0.40 | mL |
| Water to | 4.00 | mL |

Yellow, clear solution, pH 5.5.

### Example 14

### A Single Dose Sealed Aluminium Sachet Containing

| | | |
|---|---|---|
| Tizanidine•HCl | 18.59 | mg |
| Methyl p-hydroxybenzoate | 12.00 | mg |
| Citrus flavor | 80.00 | mg |
| 95% Ethanol | 0.40 | mL |
| Water to | 8.00 | mL |

Yellow, clear solution, pH 5.5.

### Preparation of Composition of Intranasal Spray Solutions:

An aqueous solution of tizanidine.HCl at high concentration, 45.00 mg/mL, and pH 4.8 is tolerated by the nasal mucosa without any local side effect, i.e. redness or pain. The proposed formulations are calculated for a single puff of 50 μL/nostril.

### Example 15

| | | |
|---|---|---|
| Tizanidine•HCl | 22.90 | mg |
| Methyl p-hydroxybenzoate | 1.00 | mg |
| Eucalyptol | 4.00 | mg |
| 95% Ethanol | 0.40 | mL |
| Water to | 1.00 | mL |

Yellow, clear solution, pH 4.8

The formulation allows administering 1.00 or 2.00 mg of tizanidine base with 1 or 2 puffs.

### Example 16

| | | |
|---|---|---|
| Tizanidine•HCl | 45.80 | mg |
| Methyl p-hydroxybenzoate | 1.00 | mg |
| Eucalyptol | 4.00 | mg |
| 95% Ethanol | 0.40 | mL |
| Water to | 1.00 | mL |

Yellow, clear solution, pH 4.8

The formulation allows administering 2.00 or 4.00 mg of tizanidine base with 1 or 2 puffs.

### Example 17

| | | |
|---|---|---|
| Tizanidine•HCl | 22.90 | mg |
| Benzyl alcohol | 10.00 | mg |
| Eucalytptol | 4.00 | mg |
| Phosphate buffer pH 7.4 (0.03M) to | 1.00 | mL |

Yellow, clear solution, pH 7.4

The formulation allows administering 1.00 or 2.00 mg of tizanidine base with 1 or 2 puffs.

### Example 18

| | | |
|---|---|---|
| Tizanidine•HCl | 22.90 | mg |
| Benzyl alcohol | 10.00 | mg |
| Eucalytptol | 4.00 | mg |
| Chitosan•HCl | 10.00 | mg |
| Phosphate buffer pH 7.4 (0.03M) to | 1.00 | mL |

Yellow, clear solution, pH 7.4

The formulation allows administering 1.00 or 2.00 mg of tizanidine base with 1 or 2 puffs.

### Example 19

| | | |
|---|---|---|
| Tizanidine•HCl | 22.90 | mg |
| Benzyl alcohol | 10.00 | mg |
| Eucalytptol | 4.00 | mg |
| Cholic acid | 14.00 | mg |
| Phosphate buffer pH 7.4 (0.03M) to | 1.00 | mL |

Yellow, clear solution, pH 7.4.

The formulation allows administering 1.00 or 2.00 mg of tizanidine base with 1 or 2 puffs.

### Pharmacokinetics of Tizanidine Solutions:

Preliminary pharmacokinetic data in rabbits at the dose of 3.00 mg/kg of tizanidine hydrochloride by intranasal (IN), intramuscular (IM), and oral route (PO) were performed to test the intranasal absorption without any enhancer. Tizanidine (IN) is rapidly absorbed with a bioavailability close to that of intramuscular route (IM).

### Biological Tests of Intranasal Preparations

According to the described criteria, a solution with the following composition was prepared to be administered with a 70 μL pump for nasal delivery.

| | 100 mL | | |
|---|---|---|---|
| Ingredient | composition | Function | Quality |
| Tizanidine HCl | g 3.273 | API | USP 32 |
| Benzalkonium chloride | g 0.100 | Preservative | E.P. |
| 1,2 propandiol | g 10.000 | Co-solvent | E.P. |
| Phosphate buffer pH 5 | mL 10.000 | Buffer | E.P. |
| Sodium hydroxide 1N | mL 0.075 | pH modifier | E.P. |
| Purified water to | mL. 100.000 | Solvent | E.P. |

### Local Toxicity:

The formulation, provisional code Tizaspray, has been tested for systemic and local tolerability given intranasally three times a day for 14 consecutive days at volume of 10 or 20 μL/kg/dose (0.321 or 0.642 mg/kg) to New Zealand White Rabbit. (Accelera s.r.l., report 0126-2011-R)

### Results:

Tizaspray 32.73 mg/mL was well tolerated systemically up to the highest dose administered. Placebo for Tizaspray given with the same modality as for Tizaspray in a volume of 20 μL/kg/dose was also systemically well tolerated.

Locally, histological nasal changes were seen in all dose groups including those receiving placebo, mainly consisting of hyperplasia/metaplasia of the transitional/respiratory epithelium, inflammatory infiltration and fibroplasias/fibrosis in the lamina propria and luminal exudates. Tizaspray treatment at both dosages induced only a mild worsening of some of these lesions. In particular, hyperplasia of the respiratory epithelium, mostly overlying the septum and/or meatuses, showed a marginally increased severity in high dose animals. Additional reactive changes represented by inflammatory cell infiltration and fibroplasias/fibrosis in the lamina propria of septum and/or turbinates and meatuses, showed a marginally increase in severity and/or incidence at both dose levels.

### Human Absorption:

The formulation, provisional code Tizaspray, has been administered intranasally to twelve human volunteers according to a randomised cross-over design. The tablets marketed in Italy (Sirdalud, Novartis, 2.29 mg as tizanidine HCl, corresponding to 2 mg of tizanidine base) were used as reference drug product vs. one or two 70 μL/puff by intranasal route (one or two nostrils). The plasma levels of tizanidine were determined by LC/MS/MS over 12 hours from the administration. (Unifarm S.r.l., report of study 317/01/2011, Eudra CT no. 2011-005420-18)

### Results:

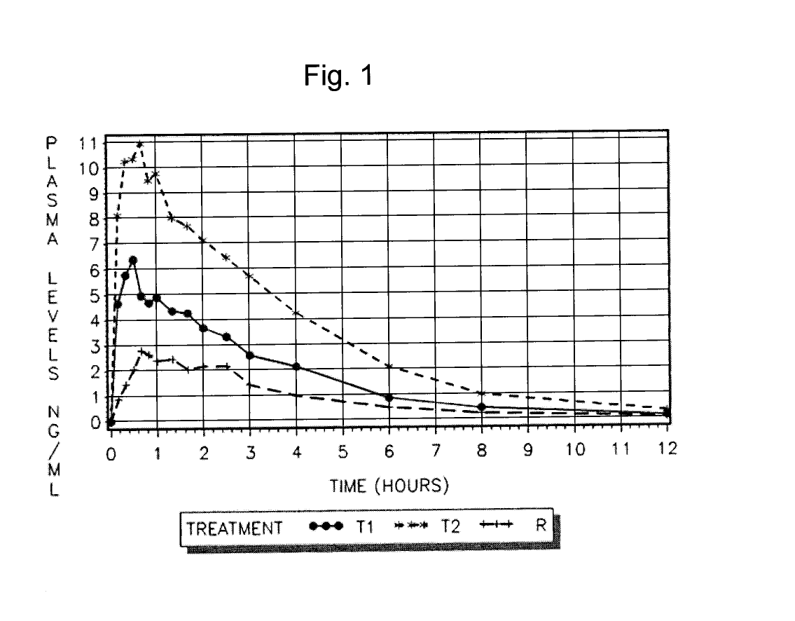

FIG. 1 shows the mean time courses up to 12 hours post-dose. R=Sirdalud tablet; T1=1*70 μL/puff by intranasal route; T2=2*70 μL/puff by intranasal route.

Mean values of main PK parameters (Mean±SD; N=12).

| | | T1 | | T2 | | R | |
|---|---|---|---|---|---|---|---|
| PARAMETERS | | | Ln | | ln | | ln |
| AUC₀₋ₜ | Mean | 19.902 | 2.834 | 40.102 | 3.616 | 9.865 | 2.174 |
| (ng · | ±SD | 9.781 | 0.50 | 15.765 | 0.418 | 4.988 | 0.509 |
| h/mL) | CV % | 49.145 | 22.931 | 39.151 | 11.555 | 50.563 | 23.398 |
| AUC_{0-inf} | Mean | 21.119 | 2.904 | 42.130 | 3.670 | 10.782 | 2.253 |
| (ng · | ±SD | 10.093 | 0.23 | 16.081 | 0.405 | 5.736 | 0.526 |
| h/mL) | CV % | 47.793 | 21.462 | 38.170 | 11.032 | 53.202 | 23.359 |
| Cₘₐₓ | Mean | 6.829 | 1.756 | 12.393 | 2.429 | 4.380 | 1.424 |
| (ng/mL) | ±SD | 3.221 | 0.681 | 5.281 | 0.455 | 1.447 | 0.349 |
| | CV % | 47.165 | 38.787 | 42.612 | 18.721 | 33.025 | 24.477 |
| tₘₐₓ | Mean | 0.431 | | 0.625 | | 1.125 | |
| (h) | ±SD | 0.207 | | 0.257 | | 0.820 | |
| | CV % | 47.975 | | 41.191 | | 72.878 | |
| | Range | 0.500 | | 0.667 | | 0.750 | |
| | Median | | | | | | |
| t_{1/2} | Mean | 2.488 | | 2.254 | | 2.042 | |
| (h) | ±SD | 0.839 | | 0.485 | | 0.916 | |
| | CV % | 33.713 | | 21.506 | | 44.878 | |
| λ_{z} | Mean | 0.304 | | 0.381 | | 0.383 | |
| | ±SD | 0.087 | | 0.056 | | 0.118 | |
| | CV % | 28.548 | | 17.467 | | 31.162 | |

### Relative Systemic Availability (Fᵣₑₗ)

| | |
|---|---|
| Arithm. mean ratio | Geom. mean ratio |

| | AUC₀₋ₜ | AUC_{0-inf} | AUC₀₋ₜ | AUC_{0-inf} |
|---|---|---|---|---|
| T1 | 19.902 | 21.119 | 17.020 | 18.250 |
| T2* | 20.051 | 21.165 | 18.595 | 19.625 |
| R | 9.685 | 10.782 | 8.790 | 9.520 |
| T1/R | 205.49% | 195.87% | 193.63% | 191.70% |
| T2*/R | 207.03% | 195.37% | 211.55% | 206.14% |
| T1/T2* | 99.26% | 100.26% | 91.53% | 92.99% |

*For T2 data are dose adjusted

The calculated PK parameters from 1n-transformed data resulted statistically significant for treatments by ANOVA.
Geometric means of transformed data:

| | | T1 | T2* | R |
|---|---|---|---|---|
| AUC₀₋ₜ | Mean | 17.02 | 18.60 | 8.79 |
| (ng · h/mL) | CV % | 72.50 | 43.68 | 54.33 |
| AUC_{0-inf} | Mean | 18.25 | 19.63 | 9.52 |
| (ng · h/mL) | CV % | 68.90 | 42.20 | 56.50 |
| Cₘₐₓ | Mean | 5.79 | 5.68 | 4.15 |
| (ng/mL) | CV % | 76.84 | 47.93 | 35.94 |

*For T2 data are dose adjusted

### ANOVA of Sources:

| | ln AUC₀₋ₜ | ln AUC_{0-inf} | ln Cₘₐₓ |
|---|---|---|---|
| Sequence | 0.4661 | 0.3531 | 0.8696 |
| Treatments | <0.0001* | <0.0001* | 0.0001* |
| Periods | 0.8909 | 0.8651 | 0.3987 |

*p < 0.05

### CONCLUSION

The formulation, provisional code Tizaspray, is absorbed by nasal route more rapidly and in higher amount than the corresponding dose by oral route.

The time to peak is about half, the peak is about double and the entity of absorption is about double of the ones by oral route.

### Discussion:

The summarized results of Tizanidine HCl solution according to the general description demonstrate:

tizanidine hydrochloride at the highest concentration is well tolerated on the nasal mucosa even after 14 days of treatment tid in rabbits.

tizanidine hydrochloride at the highest concentration is rapidly and completely absorbed by intranasal route after single administration in humans

intranasal tizanidine hydrochloride could be proposed at lower doses than the used ones by oral route in clinical practice with a lower risk/benefit ratio than the current administration.

These conclusions were not known previously and unexpected for an intranasal route.

Obviously, numerous modifications and variations of the present invention are possible in light of the above teachings. It is therefore to be understood that within the scope of the appended claims, the invention may be practiced otherwise than as specifically described herein.

1. A pharmaceutical composition suitable for oral, parenteral or intranasal administration comprising tizanidine hydrocloride dissolved in a medium selected from the group consisting of water, an aqueous saline solution and an aqueous buffer solution, wherein the composition has a pH of 4.5 to 7.5.

2. The pharmaceutical composition of claim 1, which is sterilized.

3. The pharmaceutical composition of claim 1, which is sterilized by heat treatment.

4. The pharmaceutical composition of claim 1, further comprising at least one preservative agent.

5. The pharmaceutical composition of claim 1, further comprising at least one flavoring agent, tonicity agent, hyperbaric agent or absorption enhancer agent.

6. The pharmaceutical composition of claim 1, which has a pH of from 4.5 to 6.5.

7. The pharmaceutical composition of claim 1, having a concentration of from 0.10 to 2.00 mg/mL of tizanidine base.

8. The pharmaceutical composition of claim 1, having a concentration of from 0.10 to 2.00 mg/mL of tizanidine base and a pH of from 4.5 to 6.5.

9. The pharmaceutical composition of claim 8, further comprising at least one tonicity agent or a hyperbaric agent.

10. The pharmaceutical composition of claim 1, having a concentration of from 0.10 to 1.00 mg/mL of tizanidine base.

11. The pharmaceutical composition of claim 1, having a concentration of from 0.10 to 1.00 mg/mL of tizanidine base and a pH of from 4.5 to 6.5.

12. The pharmaceutical composition of claim 11, further comprising at least one tonicity agent or a hyperbaric agent.

13. The pharmaceutical composition of claim 1, further comprising at least one preservative agent selected from the group consisting of benzyl chloride, methylparahydroxybenzoate, ethylparahydroxybenzoate and propylparahydroxybenzoate and at least one flavoring agent selected from the group consisting of aroma consisting of citrus, eucalyptol, eucalyptus oil and peppermint.

14. The pharmaceutical composition of claim 13, further comprising at least one tonicity agent and/or hyperbaric agent.

15. The pharmaceutical composition of claim 1, having a concentration of from 0.10 to 45.0 mg/mL of tizanidine base.

16. The pharmaceutical composition of claim 1, having a concentration of from 0.10 to 45.00 mg/mL of tizanidine base and a pH of from 4.5 to 6.5, and wherein the composition contains saline, glucose or a mixture thereof.

17. The pharmaceutical composition of claim 1, having a concentration of from 20.0 to 45.0 mg/mL of tizanidine base.

18. The pharmaceutical composition of claim 1, having a concentration of from 10.0 to 40.0 mg/mL of tizanidine base and a pH of from 4.5 to 7.4, and wherein the composition further comprises at least one absorption enhancer agent selected from the group consisting of chitosan, methylpyrrolidone and cholic acid.

19. The pharmaceutical composition of claim 18, which contains chitosan.

20. A method of making the pharmaceutical composition of claim 1, comprising dissolving tizanidine hydrocloride in the medium.

21. A method of delivering tizanidine, comprising orally, parenterally or intranasally administering the pharmaceutical composition of claim 1 to a subject.

22. A method of treating muscle spasms, comprising administering an effective amount of the pharmaceutical composition of claim 1 to a subject in need thereof.

23. The method of claim 22, wherein the pharmaceutical composition is administered orally, parenterally or intranasally.
